(19) United States
(12) Patent Application Publication
(10) Pub. No.: US 2005/0106122 A1
(43) Pub. Date: May 19, 2005

(54) Absorption enhancing agent

(76) Inventors: Sveinbjorn Gizurarson, Reykjavik (IS); Sigridur Olafsdottir, Revkjavik (IS); Jakob Lindal Kristinsson, Reykjavik (IS); Kolbrun Hrafnkelsdottir, Reykjavik (IS); David Rurik Olafsson, Reykjavik (IS); Oddur Ingolfsson, Reykjavik (IS); Ellen Ruth Ingimundardottir, Mosfellsbaer (IS)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/505,116

(22) PCT Filed: Feb. 24, 2003
(86) PCT No.: PCT/IS03/00010

(30) Foreign Application Priority Data
Feb. 25, 2002 (IS) 6283
Jul. 3, 2002 (IS) 6453

(51) Int. Cl. A61K 31/765; A61K 31/225

(52) U.S. Cl. 424/78.38; 514/548

(57) ABSTRACT

One or more mono- or diglyceride having the formula: (Formula I); wherein R1, R2, And R3 are selected from the group consisting of from C6 to C26 fatty acids, PEG polymers and hydrogen, provided that at least one R1, R2 and R3 is a C6-C26 fatty acid residue and at least one of R1, R2 and R3 is a PEG polymer residue for use as an absorption enhancing agent.

### FIELD OF THE INVENTION

The present invention relates to pharmaceutical compositions comprising one or more mono- and diglycerides according to formula I as an absorption enhancing agent, a bioavailability improving agent and/or a bioadhesive agent. The pharmaceutical compositions are especially suitable for mucosal administration such as intranasal administration.

### BACKGROUND OF THE INVENTION

Parenteral administration (intravenous, intramuscular and subcutaneous) of active substances, such as drugs, is normally regarded as the most effective route, of administration. However, administration by injection has a number of disadvantages. Injection of an active substance requires the use of sterile syringes and administration by trained personnel, and may cause pain and irritation, particularly in the case of repeated injections. This route of administration poses a risk of infection. More significantly, intramuscular injections are often poorly tolerated by the individual, and may possibly cause an induration (hardening of tissue), haemorrhage (bleeding) and/or necrosis (local death of tissue) at the injection site.

The mucosal membrane is connected to an extensive network of blood capillaries under the nasal mucosa, which makes the membrane highly suitable for drug delivery (delivery of active substances), particularly suited to provide rapid absorption of active substances, providing a rapid pharmacological response. One example of such a mucosal membrane is the nasal epithelial membrane, which consists essentially of a single layer of epithelial cells (pseudo-stratified epithelium), the mucosal membrane is therefore very suitable for drug delivery.

A variety of vehicle systems for intranasal drug delivery have been developed. One of the problems encountered in using such vehicle systems, is the local irritation and lack of rapid absorption. Without the rapid rate of absorption, the active substances, such as drug substances, may be cleared from the absorption site before they are absorbed into the systemic circulation, into the lymphatic system or into the brain, whichever is relevant.

It is contemplated that a vehicle that is bioadhesive as well as water-soluble would be an advantage for mucosal administration. However, to the best of the present inventors knowledge no such vehicle has yet been found. Thus, there is a need for developing a bioadhesive agent, which may be used especially in hydrophilic compositions. It is also an advantage if it can be used in lipophilic compositions as well. The bioadhesive agent should be suitable for mucosal delivery of active substances such as drug substances and/or vaccines.

A variety of vehicle systems for intranasal drug delivery have been developed. One of the problems encountered in using such vehicle systems, is the local irritation and malabsorption. A frequent problem is that the substance may be cleared from the absorption site before it may be absorbed into the systemic circulation, into the lymphatic system or into the brain. Many excipients such as polyethylene glycol and glycofurolum (Bechgaard, Gizurarson & Hjortkjaer, DK-1170/90 and Bechgaard, Gizurarson & Hjortkjaer, U.S. Pat. No. 5,397,771) are highly viscous and therefore not suitable for the purpose. Thus, there is a need for an effective formulation for intranasal or mucosal drug delivery that may be used in low concentration without being affected by the viscosity.

WO 99/02186 describes antigen delivery systems comprising monoglyceride or diglyceride derivatives as adjuvants. The monoglyceride or diglyceride derivatives mentioned therein may contain a PEG polymer. However, there is no mention or indication that such derivatives are capable of promoting absorption of an active substance to obtain an increased and/or improved onset of action or to improve the bioavailability of an active substance. Furthermore, there is no disclosure mentioning or indication that such derivatives have an ability to adhere to a mucosal surface.

U.S. Pat. No. 6,326,401 relates to a formulation for the oromucosal in particular the pemasal route. It describes pharmaceutical compositions comprising caprylcaproyl-macrogol glycerides for delivery of non-polypeptidic active substances. There is no mention or indication that the glycerides have bioadhesive properties.

The present invention provides pharmaceutical compositions, which—when administered to the mucosa such as intranasally to the nasal mucosa—enable the active substance rapidly to be absorbed into the circulatory system. The compositions comprise an absorption enhancing, a bioavailability improving and/or a bioadhesive agent that is a glyceride ester formed by esterification of glycerol with one or more polyethylene glycols and with one or more fatty acids. The absorption enhancing, bioavailability improving and/or bioadhesive agent does not irritate the nasal mucosa in the concentrations claimed.

### SUMMARY OF THE INVENTION

The present invention is based on the observation that mono- and diglycerides according to formula I are absorption enhancing agents, bioavailability improving agents and/or bioadhesive agents. Furthermore, the mono- and diglycerides are water-soluble which means that the bioadhesive properties are present in a monophasic system such as an aqueous based system. This observation of the above-mentioned effects is extremely valuable in respect of design of pharmaceutical composition for administration to mucosal surfaces. Firstly, it is possible to ensure that the composition after administration will remain on the administration site for a prolonged period of time (i.e. a rapid clearance effect can be avoided) and, secondly, due to the water-solubility of the glycerides it is possible to formulate the compositions as a single phase composition, i.e. it is not necessary to add any surfactants (including emulsifiers) etc in order to stabilize a two or more phase composition and thereby it is possible to avoid any irritating effect arising from such surfactants. Moreover, it is envisaged that it is possible to obtain an enhanced onset of action and/or a controlled delivery of the active substance to the systemic circulation or to the site of action.

Thus the present invention relates to the use of a composition comprising
one or more mono- or diglyceride having the formula (I): 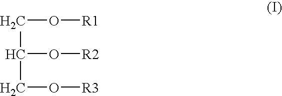
   wherein R1, R2, and R3 are selected from the group consisting of from C6 to C26 fatty acids, PEG polymers and hydrogen,
provided that at least one of R1, R2 and R3 is a C6-C26 fatty acid residue and at least one of R1, R2 and R3 is a PEG polymer residue
as an absorption enhancing, a bioavailability improving and/or a bioadhesive agent.

The term “PEG-C6/C26 glycerides” as used in the present context refers to those reaction products derived from the co-reaction of polyoxyethylene glycol (or polymerizable precursor thereof, such as ethylene oxide) with a C6-C26 carboxylic acid and glycerol or a C6-C26 carboxylic acid glyceride or glycerides. Resulting from such reactions are, typically, mixtures of a polyoxyethylene glycol C8-C10 carboxylic acid di/tri-glyceride esters (e.g.; PEG-glycerol-caprate, PEG-glycerol-caprylate etc.) as principal components.

In another aspect, the invention relates to a pharmaceutical composition for nasal administration comprising
i) one or more mono- or diglyceride having the formula (I): 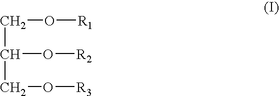
   wherein R1, R2, and R3 are selected from the group consisting of from C6 to C26 fatty acids, PEG polymers and hydrogen,
provided that it contains at least one C6-C26 fatty acid and at least one PEG polymer,
ii) a therapeutically, prophylactically and/or diagnostically active substance; and
iii) optionally, a physiologically acceptable vehicle.

Other aspects of the invention relates to a method for obtaining a relatively fast onset of a therapeutic effect or for improving the bioavailability of an active substance, the method comprises administering to a mammal including a human an efficient amount of a composition according to the invention.

### DETAILED DISCLOSURE OF THE INVENTION

As appears from the above, the present invention concerns the use of compositions comprising one or more mono-or diglycerides of formula I especially for administration to a mucosal surface. The invention is based on the observation that specific glycerides can act as absorption enhancers, bioavailability improving agents and/or bioadhesive agents and at the same time they are non-irritating to the nasal mucosal.

Thus, the present invention relates to the use of a composition comprising
one or more mono- or diglycerides having the formula (I): 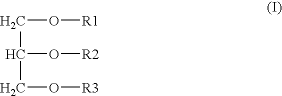
   wherein R1, R2, and R3 are selected from the group consisting of from C6 to C26 fatty acids, PEG polymers and hydrogen,
provided that at least one of R1, R2 and R3 is a C6-C26 fatty acid residue and at least one of R1, R2 and R3 is a PEG polymer residue
as an absorption enhancing, a bioavailability improving and/or a bioadhesive agent.

The PEG polymer on the glycerides imparts the desired water-solubility of the bioadhesive agent. Thus, it the water-solubility of the bioadhesive agent is relatively high, i.e. at least 40% w/w such as at least 50% w/w or at least 60% w/w. As mentioned above the high water-solubility makes it possible to design pharmaceutical compositions in liquid form that are in the form of single phase aqueous systems and thereby it is possible to avoid side effects related to the presence of surfactants or other additives that stabilize e.g. emulsions or suspensions.

Any saturated or unsaturated C6-26 fatty acid can be used including, but not limited to, fatty acid residues derived from capric acid, capric acid, caprylic acid, arachidonic acid, propionic acid, lauric acid, myristic acid, palmitic acid, oleic acid, linoleic acid and linolenic acid. Examples of suitable fatty acids are saturated or unsaturated C6-20, C6-C18, C6-14, C6-12, C8-12 or C8-10 fatty acids. In one embodiment, the fatty acids are C6, C8 or C10 fatty acids.

The fatty acid residues may be a single residue or a mixture of two or more residues. They can be derived from natural or synthetic sources, such as fats and oils.

Commercially available glycerides can be used or they can be synthesized enzymatically by means of lipases, including both specific and non-specific lipases, which place the fatty acids on specific positions (R1, R2 and/or R3) such as R1; R2; R₁, R2; R1, R3; and specific racemic structures as well, etc. For example, glycerol (0.92 g) adsorbed onto 1 g silica gel is suspended with 20 ml tBuOMe. Vinyl caprylate (3.4 g) and lipase (50 mg) from Rhizopus delemar were added to the suspension. The mixture was stirred at room temperature for 96 hours and the reaction progress was monitored by means of TLC. After removal of the solid components by filtration and evaporation of the solvent, a crude reaction mixture was obtained which contained about 91% of 1,3 dlcaprylin glyceride.

The absorption enhancing, a bioavailability improving and/or a bioadhesive agent can be prepared by oligomerizing or polymerising ethylene oxide in the presence of an ester of glycerol and one or more of such C6-C26 carboxylic acids (glyceride esters). Still another route is by co-reacting a carboxylic acid glyceride ester or esters with a fully pre-formed polyoxyethylene glycerol under conditions to achieve alcoholysis. The term “carboxylic acid glyceride ester”, is employed in this description in the conventional sense to mean an ester which has been derived from glycerol and a carboxylic acid.

A suitable PEG polymer is one that is biocompatible with the tissue to which it is administered, particularly the mucus membranes. Suitable PEG polymers having these properties include but are not limited to, polyethylene glycol, polyoxyethylene glycol, polyethylene glycol derivatives including, but not limited to, amino-PEG, nucleophilic-PEG, PEG-thiol, PEG-succinate, PEG-succinimide, PEG-tresylate, carboxymethylated-PEG, PEG-propionic acid, PEG-silanes, PEG-phospholipids, biotin-PEG and PEG-orthopyrldyl-disulfide) and polyoxyethylene glycol derivatives.

The polyethylene glycol (PEG or PEO) component used in the formation of the absorption enhancing, bioavailability improving and/or adhesive agent is, typically, a medium to high molecular weight material having a molecular weight of from about 200 to about 1200 such as, e.g. from about 300 to about 600.

Preferred are polyethylene glycol polymers having from about 2 to about 30 polyethylene glycol units (PEG2-30) or PEG polymers that have from 2 to about 15 residues such as, e.g., from 2 to about 10 residues, from 2 to about 8 residues or from about 3 to about 6 residues.

One or two PEG moieties can be incorporated into the glyceride formula. The water-soluble moiety can reside at any one of the R1, R2, and/or R3 positions of the glyceride.

Thus, the composition for use according to the invention may comprise a mixture of at least a first a second glyceride, the first glyceride having one PEG polymer in position R1, R2 or R3, and the second glyceride having two PEG polymers in position R1, R2 and/or R3.

The polymers can be attached to the glyceride via covalent bonds formed chemically or enzymatically. The polymers may be acylated to the glyceride or linked using ester bonds such as, for example, by esterase-mediated synthesis. For example, solketal can be mixed with polymerchloride in triethanolamine and trichloromethane, whereafter the free glycerol bonds are deprotected by heating in dilute aqueous acetic acid. With excess of caproyl chloride in the presence of triethylamine and 4-dimethylaminopyridine as catalyst, the fatty acids are linked to the free glycerol bonds. The results of this synthesis will be a caproyl/polymer glyceride.

In one embodiment, the composition for use according to the invention comprises a mixture of mono- and diglycerides having formula I, such as a mixture of one or more monoglycerides and/or a mixture of one or more diglycerides. The v/v-% ratio of monoglycerides to diglycerides being from about 0.1:99.9 to about 99.9:0.1 such as, e.g., from about 1:99 to about 99:1, from about 2.5:97.5 to about 97.5:2.5, and preferably from about 5:95 to about 95:5.

In a second embodiment, the mono- or diglyceride has a structure selected from the group consisting of:

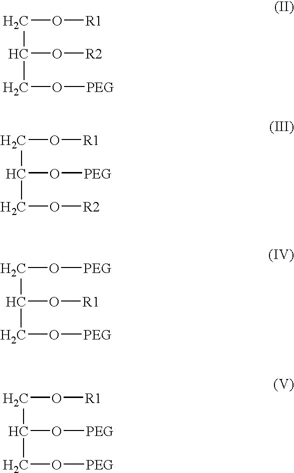

The PEG polymers in formulas II-V have from about 2 to about 30 polyethylene glycol units (PEG2-30). In a specific embodiment, the PEG in formula II-V is selected from the group consisting of PEG2, PEG3, PEG4, PEG5, PEG6, PEG7, PEG8, PEG9, PEG10, and In yet another specific embodiment, the PEG in formula II-V is PEG3 or PEG6.

In another embodiment, the monoglyceride has the following structure 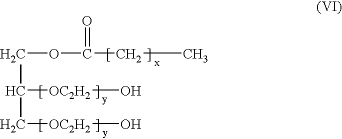
and in yet another embodiment, the diglyceride has the following structure

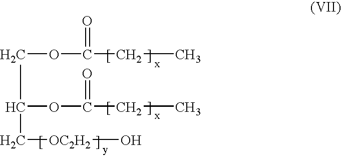

In a specific embodiment, the composition contains a mixture of the two following structures

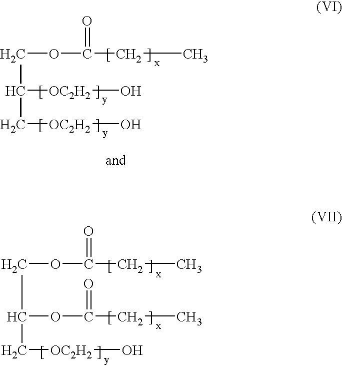

In the formulas VI, and VII, x is from about 4 to about 20, such as e.g. from about 4 to about 12, or from about 6 to about 8, and y is from 2 to about 30, such as e.g. from 2 to about 10, or from 3 to about 6. In a specific embodiment of the invention, x is 6 and/or 8 and y is 3 and/or 6.

The total concentration of glycerides of formula I in the composition is at least about 90% w/w such as, e.g., at least about 92.5% w/w, at least about 95% wlw, at least about 97.5% w/w, at least about 98% w/w or at least about 99% wlw and is normally from about 0.05-99.95 of glyceride fatty acid diester of formula II and III such as, e.g. from about 5 to about 95% glyceride fatty acid diesters of formula II and III and from about 0.05 to about 99.95% glyceride fatty acid mono esters of formula IV and V such as, e.g. from about 5 to about 95% glyceride fatty acid monoesters of formula IV and V. The fatty acid esters at the glycerides may contain about 30-90% octanoic acid esters (C8) and about 5-80% decanolc acid esters (C10).

In one embodiment, the glyceride has a chiral carbon, and in a specific embodiment the chiral carbon is S- or R-form.

Suitable absorption enhancing, a bioavailability improving and/or bioadhesive agents for use in this invention, which are commercially available, are Softigen® 767, produced by Condea Chemie GmbH (Witten, Germany). Softigen® 767 contains following specifications:

| Specification | Value |
|---|---|
| Acid value | ≦1 mg KOH/g |
| Saponification value | 90-100 mg KOH/g |
| Iodine value | ≦1 mg l/100 mg |
| Colour | ≦150 APHA |
| Freeze test | Clear solution at 0° C. (24 h) |
| Water content | max. 0.5% (Carl Fisher test) |
| Viscosity | 150-175 mPa · s |
| Refractive index | 1.464-1.466 ⁿD₂₀ |

EP-0351651 describes the use of PEG-C8/C10-glycerides as an absorption promoter for insulin. Especially for orally and buccally administered insulin. From the disclosure it appears that an increase in concentration of PEG-C8/C10-glycerides results in an increase in absorption. With respect to a nasal composition the composition described has a relatively high concentration of absorption enhancer, namely about 50% w/w.

The present invention provides glycerides according to formula I, which can be used as an absorption enhancing, a bioavailability improving and/or a bioadhesive agent in the concentrations claimed in the appended claims. It is especially interesting that the present inventors have observed that also very low concentrations lead to a suitable therapeutic response. Thus, even concentrations in a range corresponding to from 0.005 to about 2.5%, or from 0.01 to 2.0% v/v are suitable for the administration of active substances, such as drugs, through the mucosal membrane such as the nasal membrane. This agent is fully water-soluble and produces a non-viscous solution together with water or saline. The agent of the present invention provides enhanced absorption of the active substance through the nasal mucosal membrane. Use of the invention provides the ability to achieve a significant controllable systemic absorption of active substances such as drugs, into the systemic circulation, without causing unacceptable irritation of the epithellal membrane.

As mentioned above, the disclosure in EP-B-0 351 651 (Hoffmann-La Roche) is focused on oral and buccal insulin composition. The present invention is mainly directed to nasal compositions, but as discussed above, it has been found that even very small concentrations of glycerides according to the invention may lead to a suitable therapeutic response.

Therefore in another aspect, the invention relates to a pharmaceutical composition for nasal administration comprising
a composition comprising one or more mono- or diglycerides according to the invention,
ii) a therapeutically, prophylactically and/or diagnostically active substance; and
iii) optionally, a physiologically acceptable vehicle.

The present invention is also directed to compositions in general, in which the concentration of the absorption enhancing, bioavailability improving and/or bioadhesive agent is relatively low, namely in concentrations ranging from 0.005-2.5% v/v such as, e.g., 0.01-2% v/v of chemically modified glycerides selected from the group consisting of monoglycerides, diglycerides, and mixtures thereof, said glycerides having the formula (I): 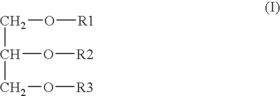
wherein R1, R2, and R3 are as defined above.

In a composition according to the present invention for nasal administration the concentration of component 1) in the composition is at the most 50% v/v such as, e.g., from about 0.005% to about 50% v/v, from about 0.005% to about 40% v/v, from about 0.01% to about 30% v/v, from about 0.01% to about 25% v/v, from about 0.01 to about 20% v/v, from about 0.01 to about 15% v/v, from about 0.01 to about 10% v/v. Alternatively, the concentration of component i) in the composition is at the most about 10% v/v such as, e.g., at the most about 7.5% v/v, at the most about 5% v/v, at the most 2.5% v/v.

Irrespective of the route of administration, the invention relates to a composition having a concentration of component i) in the composition is from about 0.01% to about 2% v/v such as, e.g. from about 0.1 to about 1.5% v/v such as from about 0.2% to about 1% v/v.

As mentioned above, a composition according to the invention leads to a relatively fast onset of the active substance contained in the composition. Thus, tₘₐₓ—when nasally administered—takes places relatively fast after administration compared to tₘₐₓ obtained after administration of a similar composition containing saline instead of component i). In other words, in a composition according to the invention, the mono- or diglycerides are used as an absorption enhancing, a bloavallablilty improving and/or a bioadhesive agent, providing a desired tₘₐₓ.

A further advantage of a composition according to the invention is that an increase in bioavailability is obtained. Thus,—when nasally administered—the bioavailability is increased with a factor of at least 2 such as, e.g., at least 5, at least 10, at least 20, at least 40 compared with the bioavailability obtained after administration of a similar composition containing saline instead of component i).

In a particular embodiment, a composition according to the invention contains the active substance ii) is in a dissolved form.

In addition to the above-described effects, the composition of the invention mediates adhesion of active substances to the nasal mucosa and thereby prolongs the residence time of the substance at the administration site. Furthermore the composition of the invention promotes absorption of active substances, such as drugs, across the nasal membrane, that is, the drug is capable of being absorbed into the systemic circulation with sufficient speed and quantity to be biologically active. This can be accomplished, since an absorption promoter increases the rate of absorption allowing a rapid onset of the drug and increases the amount absorbed across the nasal membrane.

A composition according to the invention may further comprising one or more components selected from the group consisting of: surfactants, water absorbing polymers, substances which inhibit enzymatic degradation, alcohols, organic solvents, oils, pH-controlling agents, solubilizers, stabilizers, HLB-controlling agents, viscosity controlling agents, preservatives, osmotic pressure controlling agents, propellants, air displacement, water, and mixtures thereof.

For intranasal administration, an active substance must be applied to the mucosa in such a manner that it is able to penetrate or be absorbed through the mucosa into the systemic circulation before it is washed away by the nasal secretions or cilary beat clearance. In order to penetrate the mucus, the delivery vehicle must have a certain degree of biocompatibility with the mucus membrane and hence have a certain degree of hydrophilicity and hydrophobicity. Work described herein relates to the utility of compositions described herein as absorption enhancing, bioavailability improving and/or bioadhesive agents. Accordingly, this invention also pertains to compositions, e.g. drug compositions, comprising an active substance and an absorption enhancing, bioavailability improving and/or bioadhesive agent containing 0.01-2% v/v of glycerides selected from the group consisting of PEG-C8/C10-glycerides.

The PEG-C8/C10 glycerides are products derived from the co-reaction of polyoxyethylene glycol (or poly merizable precursor thereof, such as ethylene oxide) C6-C26 carboxylic acid glyceride or glycerides or by oligomerizing or polymerising ethylene oxide in the presence of an ester of glycerol and one or more of such C6-C26 carboxylic acids (glyceride esters). Resulting from such reactions are typically mixtures of a polyoxyethylene glycol-C6-C26 carboxylic acid mono-/di-glyceride esters (e.g.; PEG-C8/C10-glycerol-dicaprate, diPEG-glycerol-caprate, PEG-glycerol-dicaprylate etc.) as principal components.

The absorption enhancing, bioavailability improving and/or bioadhesive agents of the invention mediates the adhesion of an active substance to a mucosal surface and also modulates the absorption of the substance such as a drug in order to generate successful absorption and absorption rate into the systemic circulation.

In this invention, an active substance is combined with one or more glycerides according to this present invention, and this formulation can be used to mediate adhesion of the substance to a mucosal surface of a subject such as a mammal and elicit absorption of the drug into the subject. Examples of active substances and drugs are anti-emetics and anti-nausea and drugs for the treatment of motion sickness such as ondansetron, granisetron, tropisetron, scopolamine, metopimazine; anti-migraine drugs such as sumatriptan, zolmitriptan, naratriptan, rizatriptan, eletriptan, almotriptan, frovatriptan; sex hormones, such as androgen hormones e.g testosteron and derivatives thereof; anti-narcotic treatment drugs such as nicotine, hormones such as calcitonin, drugs for the treatment of erectile dysfunction such as apomorphine, sildenafil, drugs for pain management such as morphine, drugs for sleep induction such as melatonin and the benzodiazepines, drugs for sedation, preanaestesia and treatment of epileptic seizures from the group of benzodiazepines such as diazepam, alprazolam, flunitrazepam, lorazepam, triazolam, nitrazepam, lormetazepam, midazolam, desmethyidiazepam, flurazepam; drugs for ntithromobotic treatment such as heparin, dalteparin, enoxaparin and tranexamic acid; drugs for fertility treatment such as chorlogonadotropin, menotropin, follitropin alpha, foilitropin beta and lutropin alpha.

Other active substances do include but are not limited to materials having antiviral, antiprion, antibacterial, antineoplastic, antiparasitic, anti-inflammatory and/or antifungal activity. They may act as neurotransmitter, neuromodulators, hormone, hormone releasing factor, hormone receptor agonist or antagonist. The substance may also be an activator or inhibitor of a specific enzyme, an antioxidant, a free radical scavenger or a metal chelating agent. The active substance may further be any substance which is capable of acting as a stimulant, sedative, hypnotic, analgesic, anticonvulsant, antiemetic, anxiolytic, antidepressant, tranqulilliser, cognition enhancer, agents preventing or healing amnesia, metabolic stimulator or inhibitor, appetite stimulator or inhibitor and/or narcotic antagonist or agonist. The substance may furthermore be any bioactive material found to be deficient in conjunction with the disorder being treated or prevented, for example, nutrients such as glucose, ketone bodies, and the like, or metabolic precursors such as lecithin, choline or acetyl coenzyme A for producing neurotransmitters for the treatment of Alzheimer's disease or insulin for the treatment of obesity and diabetes. The substance may also be an antibody for the treatment of viral, bacterial, prion, parasitic infections or tumours and/or cancer or for diagnosis of diseases or disorders where polyclonal or monoclonal antibodies and/or with biochemical markers characteristic of the diseases or disorder are used. Such diagnostic antibodies may be labelled with any labelling agent who may be suitable according to the invention. Gene manipulated micro-organisms may also be used for the treatment of tumours and/or cancer. The active substance may also comprise of substances selected from the group consisting of adrenal hormones, corticosteroids and derivatives, amino acids, anorectics, antibiotics, anti-allergic agents, antibodies, anti-cholinergic agents, anti-depressants, anti-epileptica and anti-spasmolytica, anti-histaminic agents, anti-hypertensive agents, antiinflammatory agents (enzymatic or non-steroidal or steroidal, anti-neoplastic agents, antiseptics, anti-tumor, anti-tussive expectorant (asthamtic agents), anti-viral and anti-cancer agents, beta-adrenergic blocking agents, blood factors such as factor VII, factor VIII etc, metabolism controlling agents, bone-metabolism controlling agents, bronchodilators, cardiotonics, cardiovascular regulatory hormones, chemotherapeutic agents, CNS-stimulants, diagnostic drugs, dopaminergic agents, enzymes, gastrointestinal hormones, hypothalamus hormones and derivatives, hypotensives, local anesthetics, migraine treatment substances, narcotics, antagonists and analgetics, pancreatic hormones and derivatives, parasympathomimetics, parasympatholytics, Parkinson's disease substances, pituitary gland hormones and derivatives, prostaglandines, protease inhibitors, sedatives, sex-hormones, sympathomimetics, thyroid gland hormones and derivatives, tranquillisers, vasoconstrictors, vasodilators, and vitamins.

The active substance such as drugs may be used in a particulate form or dissolved. The formulation is especially suitably for dissolved drugs.

The absorption enhancing effect of an absorption enhancing, bioavailability improving and/or bioadhesive agent according to this invention can be monitored with methods known in the art, such as HPLC, LC-MS, LC-MS-MS, GC, GCMS, spectroscopy and/or ELISA assays. As used herein, “absorption enhancing effect” is intended to mean the ability to increase and/or accelerate the absorption of an active substance into the systemic circulation. Absorption enhancing effect includes, but is not limited to, the ability to enhance the absorption of the active substance by increasing the transport of the substance across the nasal mucosal membrane and to accelerate this transport.

Typically the administration of the absorption enhancing, bioavailability improving and/or bioadhesive agent of the invention will also cause or result in an enhanced pharmacological response to the active substance of interest. In this context, “enhancing” is intended to mean that the absorption of an active substance is quantitatively greater and/or qualitatively better in the presence of the absorption enhancing agent than in the absence of the absorption enhancing agent. Furthermore, the absorption dynamics of the active substance can be controlled through the concentration of the absorption enhancer, to tailor the pharmacokinetics to achieve the optimal biologically active response.

Comparisons of absorption in the presence and absence of the absorption enhancing agent can be performed by routine methods, such as titres comparisons by HPLC, LC-MS, LCMS-MS, G,C, GC-MS, spectroscopy or ELISA assays, and appropriate controls. The enhanced absorption can be a result of a direct effect on the mucosal membrane or due to a more advantageous presentation of the biologically active agent to the mucus membrane.

The bioadhesive effect of the absorption enhancing, bioavailability improving and/or bioadhesive agents according to the invention, with a particular active substance, can be assessed using methods known in the art, such as bioadhesive meter, radioactive tracers, fluorescence tracers. As used herein, “bioadhesive effect” is intended to mean the ability to increase the duration of an active substance at the site of absorption. Bioadhesive effects include, but are not limited to, the ability to prolong the duration of the active substance by decreasing the clearance of the active substance from the site of absorption, such as the nasal cavity.

Typically the administration of the absorption enhancing, bioavailability improving and/or bioadhesive agent of the invention will also cause or result in a controlled release and prolonged pharmacological response to the active substance of interest. In this context, “prolonged” is intended to mean that the pharmacological effect of an active substance is quantitatively longer and/or therefore qualitatively better in the presence of the agent of the invention than in the absence of the agent. Comparisons of the effect in the presence and absence of the bioadhesive agent can be performed by routine methods, such as monitoring the clearance of radiolabelled tracers or fluorescence labelled tracers and an appropriate control. The prolonged duration can be a result of a biocompatibility with the mucosal environment and or direct effect on the mucosal membrane of due to a more advantageous presentation of the active substance to the mucus membrane.

The method of the present invention comprises administering to a mammal, particularly a human or other primate, a pharmacologically effective dose of a pharmaceutical composition according to the invention comprising an active substance and an absorption enhancing, bioavailability improving and/or bioadhesive agent according to the invention. The concentration of the absorption enhancing, bioavailability improving and/or bioadhesive agent ranges from high concentrations to low concentrations of about 0.01% to about 2% v/v, and more particularly from about 0.2% to about 1.5% v/v and more preferably between 0.2% and 1% v/v, will typically be effective to provide absorption enhancing, bioavailability improving and/or bioadhesive effects; however, variations in these dosage ranges will occur depending upon the active agent. Moreover, the particular dosage will depend upon the age, weight and medical condition of the mammal to be treated, as well as on the method of administration. The skilled artisan will readily determine suitable doses.

The composition according to the invention can be optionally administered in a pharmaceutically or physiologically acceptable vehicle, such as physiological or phosphate buffered saline, water, dextrose, ethanol polyols (such as glycerol or propylene glycol), and combinations thereof. The formulation according to the invention can be in admixture with a dispersing or wetting agent, suspending agent, and/or one or more preservatives. Suitable dispersing or wetting agents are, for example, naturally occurring phosphatides, e.g., lecithin, or soybean lecithin; condensation products of ethylene oxide with e.g. a fatty acid, a long chain aliphatic alcohol, or a partial ester derived from fatty acids and a hexitol or a hexitol anhydride, for example polyoxyethylene stearate, polyoxyethylene sorbitol monooleate, polyoxyethylene sorbitan monooleate etc. Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g. Avicel RC 591), methylcellulose; alginates such as, e.g., sodium alginate, etc. Suitable examples of preservatives for use in formulations according to the invention are parabens, such as methyl or propyl p-hydroxybenzoate, and benzalkonium chloride.

For application to the nasal mucosa nasal sprays or inhalation formulations are suitable compositions for use according to the invention. In a typical nasal formulation, the active substance is present in the form of a solution. The pharmaceutically acceptable vehicles and excipients and optional other pharmaceutically acceptable materials present in the composition such as diluents, flavouring agents, preservatives and the like are all selected in accordance with conventional pharmaceutical practice in a manner understood by the persons skilled in the art. After administration of a nasal formulation according to the invention, the active substance may be absorbed through the nasal mucosa.

Pharmaceutically acceptable excipients may include, antioxidants, buffering agents, preservatives, humectants and perfumes. Examples of antioxidants are ascorbic acid and derivatives thereof, tocopherol and derivatives thereof, butylated hydroxy anisole (BHA), and cysteine. Examples of preservatives are parabens, such as methyl or propyl p-hydroxybenzoate, and benzalkonium chloride. Examples of humectants are glycerin, propylene glycol, sorbitol, and urea. Examples of other excipients are edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil; and of polymers such as carmelose, sodium carmelose, hydroxypropylmethylcellulose, hydroxyethylcellylose, hydroxypropylcellulose, chitosane, pectin, xanthan gum, carragenan, locust bean gum, acacia gum, gelatin, and alginates.

Many mucosal formulations need some specialized mixture of excipients. Therefore formulations may comprise one or more surfactants and/or water absorbing polymers and/or substances which inhibit enzymatic degradation and/or alcohols, organic solvents, oils, pH-controlling agents, solubilizers; stabilisers, HLB-controlling agents, viscosity controlling agents, preservatives, osmotic pressure controlling agents, propellants, air displacement, water and mixture thereof. The surfactants may be selected from nonoxynol, octoxynol, tweens, spans sodium lauryl sulfate, sorbitan monopalmitate; water absorbing polymers may be selected from glycofurols and derivatives thereof, polyethylene glycol 200-7500 and derivatives thereof, polyvinylpyrrolidone, polyacrylic acid, propylene glycol, gelatine, cellulose and derivatives thereof, substances which inhibit enzymatic degradation may be selected from aprotinin, DFP, carbopol; oils may be selected from vegetable oil, peanut oil, coconut oil, maize oil, olive oil, sunflower oil, Miglyols; pH-controlling agents may be selected from acetic acid, hydrochloric acid, nitric acid, potassium metaphosphate, potassium phosphate, sodium acetate, ammonia, sodium carbonate, sodium hydroxide, sodium borate, trolamine; solubilizers may be selected from alcohol, isopropyl alcohol, water, glycofurol, polyethylene glycol 200-7500; stabilisers such as cyclodextrins; HLB controlling agents may be selected from Tween 20-85, Span 20- 80, Brij 30-98, acacia; viscosity controlling agents may be selected from cellulose and derivatives thereof, Tweens and derivatives thereof, polyethylene glycol and derivatives thereof, cetyl alcohol glycerine, propylene glycol, sorbitol, gelatin; preservatives may be selected from benzalkonium salt, benzyl alcohol, phenol, thimerosal, phenylmercuric nitrate, phenylethyl alcohol, chlorobutanol, cetylpyridinium chloride; osmotic pressure controlling agents may be selected from dextrose, sodium chloride, mannitol; and propellants may be selected from dichlorodifluoromethane, dichlorotetrafluoroethane, trichloromonofluoromethane and other non-ozone damaging propellants such as butane; air displacement may be nitrogen.

It is essential that the effective amount of the active substance can be administered in an appropriate volume. The volume should not exceed about 300 μl for a human subject when the composition is administered by the nasal route. A larger volume can be disagreeable to the patient and will drain out anteriorly through the nostrils or posteriorly toward the pharynx. The result is that a part of the active substance is lost from the absorption site. The volume is preferably from about 20 μl to about 125 μl and preferably administered into one nostril).

Adjustment and manipulation of established dosage ranges used with desired pharmacological responses, is within the ability of those skilled in the art. The active substances of the present invention are intended for use in the treatment of both immature and adult warm-blooded animals, and, in particular, humans, but also for diagnostic or prophylactic use.

The mucosal absorption enhancing, bioavailability improving and/or bioadhesive agent according to the invention, has a number of important implications. The agent can be used to tailor the absorption of an active substance or drug to achieve optimal concentration over time, and hence desired physiological response. More specific, variations in the concentrations of the absorption enhancing, bioavailability improving and/or bioadhesive agent even in a low concentration range of 0.01-2% can be used to manipulate the blood concentration of the drug over time. As a result, effective therapy, diagnosis or prophylactic treatment may be achieved. Additionally, the use of an absorption enhancing, bioavailability improving and/or bioadhesive agent of the invention can promote the ability of poorly absorbable substances to be transported across the mucosal membrane. It may also provide for safer drug delivery as the concentration can be controlled to meet the requirement for therapeutically concentration but may still be low enough to minimise the risk of toxic reaction. When the active substance is toxic at the concentration normally required for effective therapy. By reducing the dose, the risk of toxic reaction is reduced. It may also provide for safer drug delivery. Furthermore, the absorption enhancing, bioavailability improving and/or bioadhesive agent according to the invention can increase the duration of mucosally administered antigens in the vaccinated organism, providing appropriate time for the antigen presenting cells to recognize the antigen. This is due to bioadhesive effect of the agent. As a result, effective vaccination can be achieved with a smaller quantity of antigen than would be normally required. This reduction in the required amount of antigen may lead to more widespread use of vaccines, which are difficult and costly to prepare.

Typically, an effective serum concentration of an active substance is gained over a period of weeks or months. A clinically relevant concentration may be generated with much reduced time course by the mean of this invention. In some instances, it may result in the generation of a successful response in a single dose.

The composition according to the invention is especially suitable for humans, including toddlers, adolescents, teenagers, adults and elderly. The nature of the composition provides the ability to enhance and control the absorption of a variety of active substances and the ability to prolong the duration of residence time of the substance at the administration site, and therefore the formulation may be used for subjects with various conditions such as humans with disease, e.g., splenectomized subjects, subjects with cancer, subjects using anticancer drugs, subjects using antiasthmatic drugs, subjects using anti-inflammatory drugs, subjects with hyper- and hypothyroidea, subjects having problems with malabsorption such as diarrhoea or emesis in addition to humans with nausea or those who have problems swallowing.

The composition according to the invention is also suitable for administration to animals such as horses, sheep, dogs, cats, cows, pigs, goats, rabbits, wild animals and laboratory animals such as mice, rats, guinea pigs, hamsters, rabbits, dogs, cats or monkeys; to birds such as chickens, turkeys, ducks, ostrich, tropical birds or wild birds. For animals, the concentration of each component may need to be adjusted. For example for sheep, the nasal cavity has extremely high humidity, which may require addition of water absorbing excipients to the composition. A person skilled in the art will know how to adjust the composition and the dosage amount in order to achieve a desired effect. in a specific embodiment, the invention relates to a composition comprising a drug substance for the treatment of migraine, such as the triptans like e.g. sumatriptan; or for the treatment of emesis and nausea, such as ondansetron, or a hormone, such as testosteron.

In a further embodiment, the invention relates to a composition comprising
i) from about 0.01% to about 2.5% v/v of a mixture of 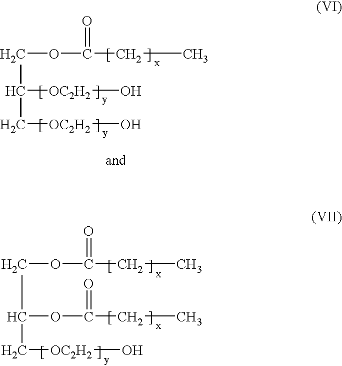
   wherein x is from about 4 to about 20, such as e.g. from about 4 to about 12, or from about 6 to about 8, and y is from 2 to about 30, such as e.g. from 2 to about 10, or from 3 to about 6.
ii) a triptan such as, e.g., sumatriptan,
iii) optionally, a physiologically acceptable vehicle, and
iv) water.

The invention further relates to a method of eliciting a therapeutic, prophylactic and/or diagnostic effect in a mammal, comprising administering to the mammal an effective amount of a composition according to invention.

In one embodiment, the invention relates to a method for obtaining a fast onset of a therapeutic, prophylactic and/or diagnostic effect of an active substance in a mammal including a human, comprising administering to the mammal an effective amount of a composition according to the invention, the onset being faster when compared with a similar composition containing saline instead of component i) and using tₘₐₓ and/or Cₘₐₓ as measures for the onset.

In a second embodiment, the invention relates to a method for improving the bioavailability of a therapeutic, prophylactic and/or diagnostic effect of an active substance in a mammal including a human, comprising administering to the mammal an effective amount of a composition according to the invention, the bioavailability being improved when compared with a similar composition containing saline instead of component i) and using AUC_{0-infinity} as a measure.

In a specific embodiment, the administration is nasal administration, and in a further embodiment, the volume to be administered to humans ranges from about 20 to about 300 μL.

Further interesting embodiments appear from the appended claims.

The following Examples are included to illustrate the present invention and are not to be construed to limit the scope of this invention. The contents of all references, patents and published patent applications cited throughout this application are hereby incorporated by reference.

### EXAMPLES

### Example 1

### Preparation of Compositions According to the invention and in Vivo Behaviour in Rabbits

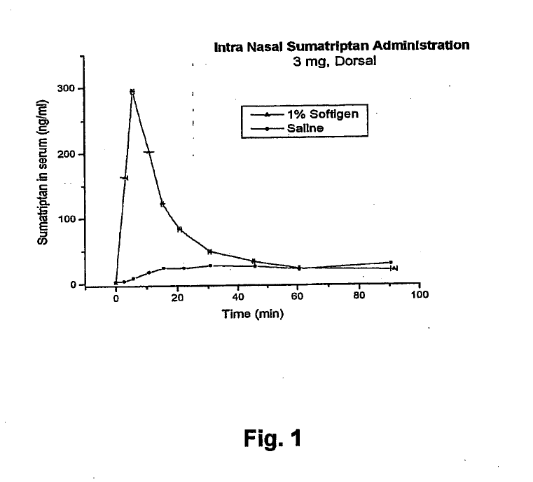

Two formulations, A and B. were made. Formulation A contains Sumatriptan, 1% Softigen 767 (PEG-C8/C10-glyceride) in water; and Formulation B contains Sumatriptan in phosphate buffered saline, as Imigran®. These formulations were administered intranasally to rabbits and the sumatriptan serum concentration was determined as a function of time. The results show a significant improvement of Cₘₐₓ, tₘₐₓ, bioavailability and clinical response when softigen was used over the use of Imigran®. FIG. 1 shows the results. Cₘₐₓ was improved from 40 ng/ml to 600 ng/ml and tₘₐₓ was improved from 30-45 min and down to 3-5 minutes.

### Example 2

### Relationship Between the Concentration of Absorption Promoting Substance and the in Vivo Behaviour

Four formulations, I, II and III, were made. Formulation I contains Sumatriptan, 1% Softigen 767 (PEG-C8/C10-glyceride) in water; Formulation 11 contains Sumatriptan, 0,5% Softigen 767 (PEG-CS/C10-glyceride) in water, Formulation III contains Sumatriptan, 0,2% Softigen 767 (PEG-C8/C10-glyceride) in water; and Formulation IV contains Sumatriptan in phosphate buffered saline, as Imigran®. These formulations were administered intranasally to rabbits and the sumatriptan serum concentration was determined as a function of time. The results show that the time to tₘₐₓ may be controlled with the use of right concentration of Softigen 767. Table 1 shows the results. There was a linear relationship between the concentration of Softigen 767 and time to tₘₐₓ.

TABLE 1

| DRUG/Formulation | tₘₐₓ |
|---|---|
| IMIGRAN | 31 min (n = 8) |
| 0.2% SOFTIGEN | 12.5 min (n = 4) |
| 0.5% SOFTIGEN | 8.5 min (n = 3) |
| 1.0% SOFTIGEN | 4.1 min (n = 7) |

### Example 3

### Demonstration of a Significant Improved Effect when Softigen® is Applied as an Absorption Promoter and a Bioadhesive Agent According to the Invention

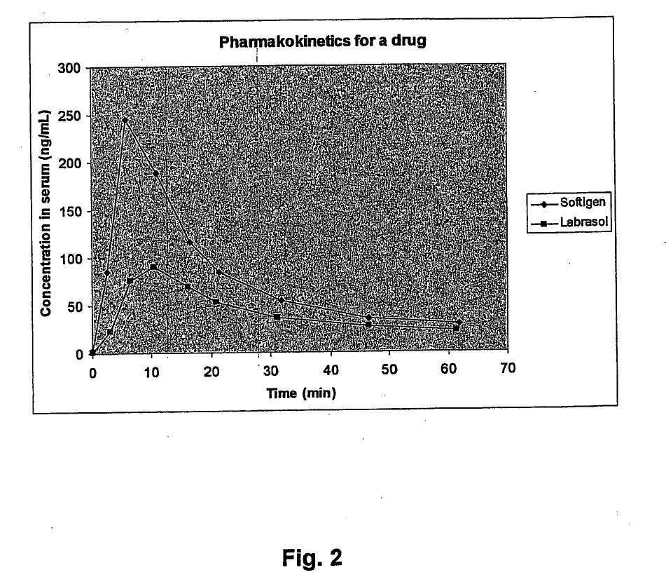

In a cross-over study, 4 rabbits received drug A in a formulation containing 1% Soitigen® (Sasol GmbH, Germany) (n=4) and 1% Labrasol® (Gattefosse, France) (n=4) and were sampled for blood samples after 0, 2, 5, 10, 15, 30, 45 and 60 min. Comparison of these two formulations showed that Softigen® had AUC=4.875 and tmax=6 min whereas Labrasol® had AUC=2.552 and tmax was 10 min. See FIG. 2.

By the manufacturer Labrasol®, is denoted caprylocaproyl macrogolglycendes that are mixtures, mainly of the following compounds

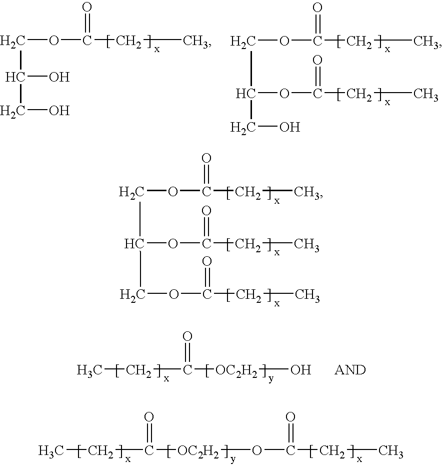

x=6 or 8 (capric and caprylic acids), y=8 on average, free glycerol is less 5%. Accordingly, Labrosol® is not a composition according to the present invention as it is not included in the definition of compounds of formula I.

In contrast to Labrasol®, Softigen is a composition according to formula I.

Softigen® is denoted macrogol-6-glycerol-caprylocaprate and is mixtures, mainly of the following compounds:

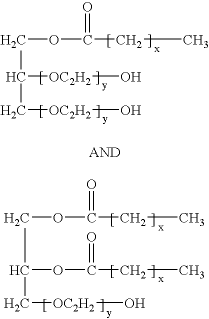

x=6 or 8 (capric and caprylic acids) and the sum of y on each molecule is on average 6.

Softigen consists of octanoic/decanoic macrogol-6 glycerides, which are made by ethoxylation of mono and diglycerides of octanoic and decanoic acids. Labrasol on the other hand is a mixture of macrogol-8 octanoic/decanoic mono or diester and mono-, di- and triesters of glycerol. This composition results from the partial alcoholysis of the corresponding triglycerides using macrogol 400.

In the following table is given a comparison of Softigen® and Labrasol®.

| Trade name | Softigen 767 ® | Labrasol ® |
|---|---|---|
| Source | Sasol GmbH | GATTEFOSSE |
| | www.condea.com | http://www.gattefosse.com |
| | Softigen 767 ® | Labrasol ® |
| Chemical | Macrogol-6-glycerol | Caprylocaproyl |
| composition | caprylocaprate is a | macrogol-8 glycerides |
| | mixture of mono and | is a well defined |
| | diesters, made of | mixture of mono-, di- |
| | polyoxyethyl glycerol | and triglycerides and |
| | ethers | mono and di-fatty acid |
| | | esters of polyethylene |
| | | glycol |
| Ph. Eur. | Macrogol-6-glycerol | Caprylocaproyl |
| | caprylocaprate | macrogolglycerides |
| | 01/2000:1443 | 01/2002:1184 |
| Average # of | Average number of | Eight (8). Applies only |
| ethoxy units | ethoxy units per | for the mono and di- |
| | molecule is six (6). | fatty acid esters of |
| | | polyethylene glycol |
| | | (PEG 400) |
| Methods of | Ethoxylation of mono and | The substance is made |
| synthesis | dl glycerides from capric- | by an |
| | and caprylic acid that are | alcoholysis/esterification |
| | synthesized through | reaction using |
| | esterification of glycerol | medium chain |
| | with destilled coconut or | triglycerides from |
| | palm-core fatty acids | coconut oil and PEG |
| | | 400 as starting |
| | | material. |
| Viscosity @ | ca. 160 mPa s | 80-110 mPa s |
| 20° C. | | |
| HPB value | ca. 19 | 14 |

1-42. (canceled)

43. A pharmaceutical composition for nasal administration, comprising:
i) a composition comprising one or more mono-or dyglyceride having the formula (I): 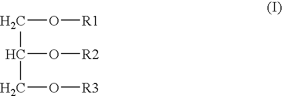
wherein R1, R2 and R3 are selected from the group consisting of from C6 to C26 fatty acids, PEG polymers and hydrogen, provided that at least one of R1, R2 and R3 is a C6-C26 fatty acid residue and at least one of R1, R2 and R3 is a PEG polymer residue;
ii) a therapeutically, prophylactically and/or diagnostically active substance; and
iii) optionally, a physiologically acceptable vehicle.

44. The pharmaceutical composition according to claim 43, wherein the PEG polymer has a molecular weight of from about 200 to about 1200.

45. The pharmaceutical composition according to claim 43, wherein the PEG contains from 2 to about 30 residues of ethylene glycol or derivatives thereof.

46. The pharmaceutical composition according to claim 43, wherein the composition comprises a mixture of at least a first and a second glyceride, the first glyceride having one PEG polymer in position R1, R2 or R3, and the second glyceride having two PEG polymers in position R1, R2 and/or R3.

47. The pharmaceutical composition according to claim 43, wherein the v/v-% ratio of monoglycerides to diglycerides is from about 0.1:99.9 to about 99.9:0.1.

48. The pharmaceutical composition according to claim 43, wherein the mono- or diglyceride has a structure selected from the group consisting of:

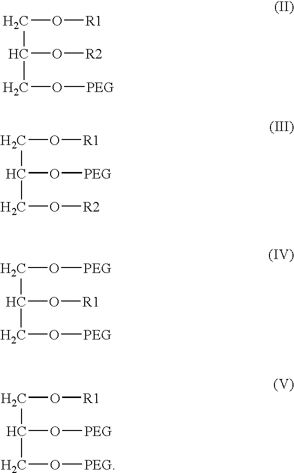

49. The pharmaceutical composition according to claim 48, wherein the PEG is selected from the group consisting of PEG2, PEG3, PEG4, PEG5, PEG6, PEG7, PEG8, PEG9 and PEG10.

50. The pharmaceutical composition according to claim 48, wherein PEG is PEG3 or PEG6.

51. The pharmaceutical composition according to claim 43, wherein at least one of R1, R2 and R3 is a saturated C6-26 fatty acids such as, e.g., saturated C6-20, saturated C6-C18, saturated C6-14, saturated C6-12, saturated C8-12 or saturated C8-10 fatty acids.

52. The pharmaceutical composition according to claim 43, wherein the glyceride has the following structure 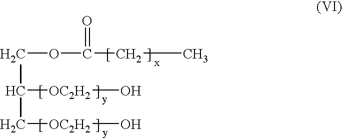
wherein x is from about 4 to about 20, e.g., from about 4 to about 12, or from about 6 to about 8; and
wherein y is from about 2 to about 30, such as e.g., from 2 to about 10, or from 3 to about 6.

53. The pharmaceutical composition according to claim 43, wherein the glyceride has the following structure 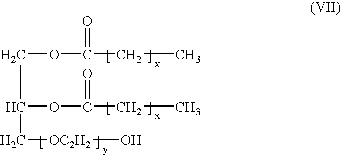
wherein x is from about 4 to about 20, e.g., from about 4 to about 12, or from about 6 to about 8; and
wherein y is from about 2 to about 30, such as e.g., from 2 to about 10, or from 3 to about 6.

54. The pharmaceutical composition according to claim 43, wherein the composition contains a mixture of 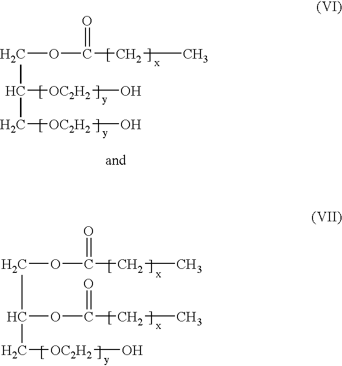
wherein x is from about 4 to about 20, e.g., from about 4 to about 12, or from about 6 to about 8; and
wherein y is from about 2 to about 30, such as e.g., from 2 to about 10, or from 3 to about 6.

55. The pharmaceutical composition according to claim 43, further comprising one or more components selected from the group consisting of: surfactants, water absorbing polymers, substances which inhibit enzymatic degradation, alcohols, organic solvents, oils, pH-controlling agents, solubilizers, stabilizers, HLB-controlling agents, viscosity controlling agents, preservatives, osmotic pressure controlling agents, propellants, air displacements, water, and mixtures thereof.

56. A pharmaceutical composition for nasal administration, comprising:
i) a composition comprising one or more mono- or dyglyceride the formula (I): 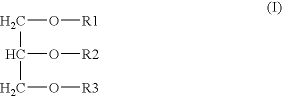
wherein R1, R2 and R3 are selected from the group consisting of from C6 to C26 fatty acids, PEG polymers and hydrogen, provided that at least one of R1, R2 and R3 is a C6-C26 fatty acid residue and at least one of R1, R2 and R3 is a PEG polymer residue;
ii) a therapeutically, prophylactically and/or diagnostically active substance, wherein the active substance is a hormone or drug substance for treatment of emesis or nausea; and
iii) optionally, a physiologically acceptable vehicle.

57. A method for obtaining a fast onset of a therapeutic, prophylactic and/or diagnostic effect of an active substance in a mammal including a human, comprising administering to the mammal an effective amount of a composition according to claim 43, the onset being faster when compared with a similar composition containing saline instead of component i) and using tₘₐₓ and/or Cₘₐₓ as measures for the onset.

58. A method for improving the bioavailability of a therapeutic, prophylactic and/or diagnostic effect of an active substance in a mammal including a human, comprising administering to the mammal an effective amount of a composition according to claim 43, the bioavailability being improved when compared with a similar composition containing saline instead of component i) and using AUC_{infinity} as a measure.
